SEQUENCE LISTING (15 sequences)

SEQ ID NO: 1 - protein, 16 aa, Trichophyton sp.
LSVAEGVGLFNVLQEK

SEQ ID NO: 2 - protein, 14 aa, Trichophyton sp.
ALVSHDGTFVGSSK

SEQ ID NO: 3 - protein, 11 aa, Trichophyton sp.
GGVGIWISDAK

SEQ ID NO: 4 - protein, 12 aa, Trichophyton sp.
INFVGYGQSTTK

SEQ ID NO: 5 - protein, 13 aa, Trichophyton sp.
TLYVTAEDHATGK

SEQ ID NO: 6 - protein, 23 aa, Trichophyton sp.
AAGAIVYNNVPGSLAGTLGGLDK

SEQ ID NO: 7 - protein, 16 aa, Trichophyton sp.
VSFGIITDNVNANLTK

SEQ ID NO: 8 - protein, 11 aa, Trichophyton sp.
LIVGFVTELAK

SEQ ID NO: 9 - protein, 14 aa, Trichophyton sp.
HANAVNAMIATLSK

SEQ ID NO: 10 - protein, 15 aa, Trichophyton sp.
KPGGTTYYYDPSAGK

SEQ ID NO: 11 - protein, 16 aa, Trichophyton sp.
MANDVIQSPGEGTTGK

SEQ ID NO: 12 - protein, 15 aa, Trichophyton sp.
VLDCDGSGSNSGVIK

SEQ ID NO: 13 - protein, 17 aa, Trichophyton sp.
ADFSNYGAVVDVYAPGK

SEQ ID NO: 14 - protein, 11 aa, Trichophyton sp.
SVMNMSLGGPR

SEQ ID NO: 15 - protein, 17 aa, Trichophyton sp.
QMAIDVIQNPGASTTSK

(19) United States
(12) Patent Application Publication
(10) Pub. No.: US 2016/0054319 A1
(43) Pub. Date: Feb. 25, 2016

(54) METHOD FOR DETECTING TRICHOPHYTONS AND ASSOCIATED DISEASES

(71) Applicant: GALDERMA RESEARCH & DEVELOPMENT, Biot (FR)

(72) Inventors: André JOMARD, Saint Vallier De Thiey (FR); Bruno MEHUL, Gourdon (FR); Olivier ROYE, Fayence (FR); Gilbert LAFFET, Biot (FR)

(21) Appl. No.: 14/654,229

(22) PCT Filed: Dec. 17, 2013
(86) PCT No.: PCT/EP2013/076939

§ 371 Date: Jun. 19, 2015

Related U.S. Application Data
(60) Provisional application No. 61/745,912, filed on Dec. 26, 2012.

(30) Foreign Application Priority Data
Dec. 21, 2012 (FR) 1262652

(51) Int. Cl. G01N 33/569; C07K 16/40

(52) U.S. Cl. CPC G01N 33/56961; C07K 16/40; G01N 2333/958

(57) ABSTRACT

A method is described for detecting the presence of at least one trichophyton involved in a mycosis of the skin and skin apendages. The method can include a step of determining the presence, in a sample of skin or skin appendage from a human being or an animal that may be infected with a trichophyton, of at least one protease selected from dipeptidyl peptidase V, subtilisin-like protease 6, subtilisin-like protease 7, leucine aminopeptidase 1 and leucine aminopeptidase 2. Also described, is a monoclonal antibody directed against dipeptidyl peptidase V, subtilisin-like protease 6 or leucine aminopeptidase 2 or a fragment of this antibody capable of binding respectively to dipeptidyl peptidase V, subtilisin-like protease 6 or leucine aminopeptidase 2, and a kit including this antibody.

The present invention relates to detection of the presence of fungi of the genus Trichophyton in humans or animals, on the skin and its appendages.

The invention relates in particular to a method for detecting Trichophytons, comprising a step of determining the presence, in a sample of skin or skin appendage, of at least one protease selected from five particular proteases. The invention also specifically relates to monoclonal antibodies directed against these proteases and a diagnostic kit containing them.

The Trichophytons are parasitic fungi that develop on the skin, the scalp or the nails and cause dermatophytoses in humans or animals. In humans, for example, the Trichophytons may be responsible for tinea circinata on the skin, tinea capitis on the scalp, kerion or trichophytic sycosis on the beard area and onychomycosis on the nails.

These diseases are very troublesome and may cause other serious pathological complications. Their unsightly and sometimes handicapping appearance may also have considerable negative psychosocial effects for the persons affected.

There are at present therapeutic means for treating the diseases caused by Trichophytons in humans or animals, but these treatments are closely linked to the soundness of the diagnosis that is made.

Now, the mycoses of the skin or its appendages may be due to fungal agents other than Trichophytons, such as yeasts, molds or other dermatophytes, and in this case a different treatment will be required. Moreover, they may be confused with other diseases or injuries of the skin or its appendages. For example, the clinical signs of dystrophy of the nails such as psoriasis, trauma, ungual tumor, lichen planus and bacterial infection resemble onychomycoses and may be sources of confusion.

It is therefore essential to be able to make an accurate diagnosis, as inaccuracy results in an ineffective therapeutic solution being initiated.

Today there are various diagnostic tools, but none is satisfactory. For onychomycosis in particular, six different tools are known (Rothmund et al., 2012), two of which are commonly used: direct observation, the uncertainty of which leads inevitably to errors of diagnosis, and culturing the pathogen, which is a long and rather impractical procedure that is not accessible to everyone. There is also an immuno-kit produced by the company NISSHO CORPORATION, which has the advantage of being quick and easy to use, but it lacks sensitivity and especially specificity. In fact, dermatophytes are detected by using an antibody that detects a polysaccharide present on the cell wall of the Trichophytons, but also on that of many other dermatophytes.

Therefore there is still a need for a solution for detecting rapidly, easily and specifically the presence of Trichophytons on the skin or its appendages, and in particular on the nails, that overcomes the drawbacks of the current diagnostic tools.

This is the aim of the present invention, which in order to meet this need proposes to use at least one protease selected from dipeptidyl-peptidase V, subtilisin-like protease 6, subtilisin-like protease 7, leucine aminopeptidase 1 and leucine aminopeptidase 2.

The invention therefore relates to the use of at least one protease selected from dipeptidyl-peptidase V, subtilisin-like protease 6, subtilisin-like protease 7, leucine aminopeptidase 1 and leucine aminopeptidase 2, for detecting the presence of at least one Trichophyton in a sample of human or animal skin or skin appendage.

In particular, the invention relates to a method for detecting the presence of at least one Trichophyton, comprising a step of determining the presence, in a sample of skin or skin appendage obtained from a human being or from an animal that may be infected with a Trichophyton, of at least one protease selected from dipeptidyl-peptidase V, subtilisin-like protease 6, subtilisin-like protease 7, leucine aminopeptidase 1 and leucine aminopeptidase 2.

The Trichophytons tested for and detected are implicated in diseases of the skin and/or of the skin appendages.

“Appendages” means, in the sense of the invention, any protective epidermal appendages, adnexa or integuments containing a high percentage of keratin, such as body hair, the hair of the head and the nails.

Advantageously, the presence of at least one of these proteases at the level of the skin or its appendages is only specific to the presence of Trichophytons. This determination may be performed by various means that may be the object of quick diagnostic tools that are easy to use, and specific to the Trichophytons, thus allowing an effective, targeted treatment to be initiated very quickly. Two means are particularly suitable: determination by mass spectrometry and immuno-detection.

To implement this second means, according to a particular aim, the invention also relates to a monoclonal antibody directed against dipeptidyl-peptidase V, subtilisin-like protease 6 or leucine aminopeptidase 2 or a fragment of this antibody capable of binding to dipeptidyl-peptidase V, subtilisin-like protease 6 or leucine aminopeptidase 2 respectively, as well as a kit comprising such an antibody.

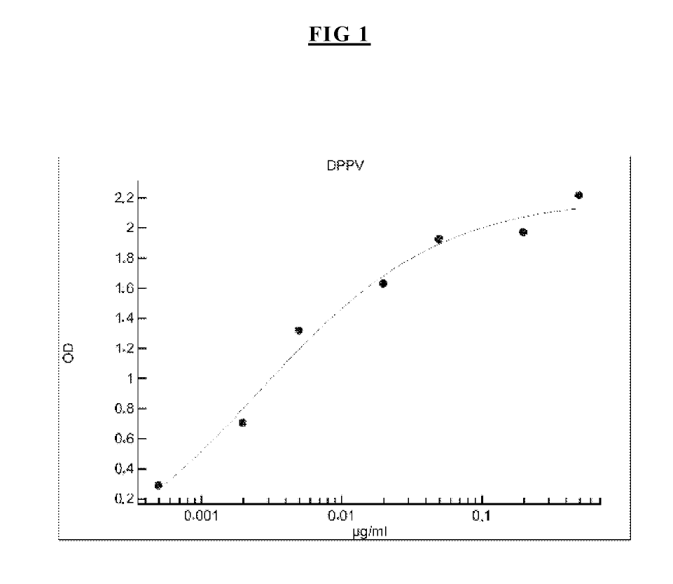

FIG. 1 describes the limit of quantitation of dipeptidyl-peptidase V.

The invention will now be described in detail.

The invention therefore relates to the use of at least one protease selected from dipeptidyl-peptidase V, subtilisin-like protease 6, subtilisin-like protease 7, leucine aminopeptidase 1 and leucine aminopeptidase 2, for detecting the presence of at least one Trichophyton in a sample of human or animal skin or skin appendage implicated in a mycosis of the skin and/or of the skin appendages.

Preferably this use consists of carrying out a method for detecting the presence of at least one Trichophyton implicated in a mycosis of the skin and/or of the skin appendages, comprising a step of determining the presence, in a sample of skin or skin appendage obtained from a human being or from an animal that may be infected with a Trichophyton, of at least one protease selected from dipeptidyl-peptidase V, subtilisin-like protease 6, subtilisin-like protease 7, leucine aminopeptidase 1 and leucine aminopeptidase 2.

Determination of the presence of at least one of the proteases is performed in vitro starting from a sample of skin or its appendages, but reflects the presence of this protease in vivo at the level of the skin or its appendages of the person or animal from whom or from which the sample was taken.

The samples used for carrying out the invention are obtained by any suitable means. When the sample is a sample of nail, it is preferably obtained by scraping the nail bed or by piercing the nail using a microdrill. This last-mentioned method of sampling is notably described in U.S. Pat. No. 7,848,799. When the sample is from a mycotic lesion of the skin, it is preferably obtained by scraping using a curette.

According to the invention, the presence, in the sample, of at least one protease selected from dipeptidyl-peptidase V, subtilisin-like protease 6, subtilisin-like protease 7, leucine aminopeptidase 1 and leucine aminopeptidase 2, is synonymous with infection with at least one Trichophyton. In fact they are proteases secreted specifically by the Trichophytons, performing a role in the digestion of keratins, and which, according to the invention, are located at the level of the skin and its appendages infected with at least one Trichophyton.

The use according to the invention is suitable for detecting any Trichophyton implicated in a mycosis of the skin and/or of the skin appendages in humans or animals, in particular Trichophyton bullosum, Trichophyton circumvolutum, Trichophyton concentricum, Trichophyton eboreum, Trichophyton equinum, Trichophyton eriotrephon, Trichophyton fischeri, Trichophyton gourvilii, Trichophyton interdigitale, Trichophyton kanei, Trichophyton krajdenii, Trichophyton longifusum, Trichophyton megninii, Trichophyton mentagrophytes, Trichophyton phaseoliforme, Trichophyton quinckeanum, Trichophyton raubitschekii, Trichophyton rubrum, Trichophyton schoenleinii, Trichophyton soudanense, Trichophyton sp. CZ-2011, Trichophyton sp. FSU 10097, Trichophyton sp. IFM 41172, Trichophyton sp. LM 10725, Trichophyton terrestre, Trichophyton thuringiense, Trichophyton tonsurans, Trichophyton vanbreuseghemii, Trichophyton verrucosum, Trichophyton violaceum and Trichophyton yaoundei, regardless of the strain if there are several.

The use according to the invention is quite particularly suitable for detecting Trichophyton interdigitale and/or Trichophyton rubrum, even more preferably Trichophyton rubrum, regardless of the strain.

Detection of the presence of Trichophytons by determining at least one protease selected from dipeptidyl-peptidase V, subtilisin-like protease 6, subtilisin-like protease 7, leucine aminopeptidase 1 and leucine aminopeptidase 2 according to the invention, may notably be used for screening, therapeutic follow-up and/or diagnosis of a disease in humans or animals, in particular of a mycosis associated with a Trichophyton infection.

The invention also relates to at least one protease selected from dipeptidyl-peptidase V, subtilisin-like protease 6, subtilisin-like protease 7, leucine aminopeptidase 1 and leucine aminopeptidase 2, for use thereof in a method of screening, of therapeutic follow-up and/or of diagnosis of a disease associated with an infection with at least one Trichophyton.

For humans, for example, it may be a question of tinea circinata, tinea capitis, kerion, trichophytic sycosis or an onychomycosis. The invention relates quite particularly to screening, therapeutic follow-up and/or diagnosis of an onychomycosis.

Thus, the invention also relates to at least one protease selected from dipeptidyl-peptidase V, subtilisin-like protease 6, subtilisin-like protease 7, leucine aminopeptidase 1 and leucine aminopeptidase 2, for use thereof in a method of screening, of therapeutic follow-up and/or of diagnosis of an onychomycosis.

“Therapeutic follow-up” means the use according to the invention and more particularly the method for detecting the presence of Trichophytons in a sample of skin or its appendages of a human being or of an animal for following the evolution of a treatment of an infection with Trichophyton.

According to a first variant, the use according to the invention, and more particularly the method for detecting the presence of Trichophytons in a sample of skin or its appendages of a human being or of an animal, comprises a step of determining the presence of at least one protease selected from dipeptidyl-peptidase V, subtilisin-like protease 6, subtilisin-like protease 7, leucine aminopeptidase 1 and leucine aminopeptidase 2, by quantitative mass spectrometry (MRM: Multiple Reaction Monitoring).

The MRM method allows the detection and quantification of a given protein in a complex mixture, using a mass spectrometer that makes it possible to target the peptides of a given protein and only detect the latter to quantify them. The mass spectrometry analyses are preferably carried out with different mass spectra essentially in MS/MS mode coupled upstream to a nano-HPLC. Assay of the peptides may also be carried out with a different (ionizing) source of the MALDI type.

According to the invention, this step consists in particular of identifying, by quantitative mass spectrometry, at least one of the peptide sequences belonging to one of said proteases, preferably one of the following peptide sequences:

SEQ ID No. 1:
LSVAEGVGLFNVLQEK
SEQ ID No. 2:
ALVSHDGTFVGSSK
SEQ ID No. 3:
GGVGIWISDAK
SEQ ID No. 4:
INFVGYGQSTTK
SEQ ID No. 5:
TLYVTAEDHATGK
SEQ ID No. 6:
AAGAIVYNNVPGSLAGTLGGLDK
SEQ ID No. 7:
VSFGIITDNVNANLTK
SEQ ID No. 8:
LIVGFVTELAK
SEQ ID No. 9:
HANAVNAMIATLSK
SEQ ID No. 10:
KPGGTTYYYDPSAGK
SEQ ID No. 11:
MANDVIQSPGEGTTGK
SEQ ID No. 12:
VLDCDGSGSNSGVIK
SEQ ID No. 13:
ADFSNYGAVVDVYAPGK
SEQ ID No. 14:
SVMNMSLGGPR
SEQ ID No. 15:
QMAIDVIQNPGASTTSK

Sequences 1 to 5 are specific to dipeptidyl-peptidase V, sequence 6 is specific to leucine aminopeptidase 2, sequences 7 to 9 are specific to leucine aminopeptidase 1, sequences 10 to 14 are specific to subtilisin-like protease 6 and sequence 15 is specific to subtilisin-like protease 7.

Detection of at least one of the peptide sequences belonging to dipeptidyl-peptidase V, subtilisin-like protease 6, subtilisin-like protease 7, leucine aminopeptidase 1 or leucine aminopeptidase 2 is specific of the presence of a Trichophyton in the sample and consequently of a disease linked to the presence of this Trichophyton.

This method makes it possible to detect the Trichophytons present with high sensitivity, high specificity, and high selectivity. According to another advantage, it also makes it possible to quantify the proteases detected and consequently quantify the Trichophytons present in the sample.

According to another variant, the use according to the invention and more particularly the method for detecting the presence of Trichophytons in a sample of skin or its appendages of a human being or of an animal, comprises carrying out an immunologic test for determining the presence, in the sample, of at least one of the proteases selected from dipeptidyl-peptidase V, subtilisin-like protease 6, subtilisin-like protease 7, leucine aminopeptidase 1 or leucine aminopeptidase 2, preferably at least one of the proteases selected from dipeptidyl-peptidase V, subtilisin-like protease 6 and leucine aminopeptidase 2.

Particularly suitably, this immunologic test comprises the use of at least one monoclonal antibody directed against dipeptidyl-peptidase V, subtilisin-like protease 6 or leucine aminopeptidase 2 or a fragment of this antibody capable of binding respectively to dipeptidyl-peptidase V, subtilisin-like protease 6 or leucine aminopeptidase 2.

This antibody may notably be used for detecting and identifying the presence of dipeptidyl-peptidase V, subtilisin-like protease 6 or leucine aminopeptidase 2 by an immuno-detection system, for example a liquid-phase immuno-detection (ELISA for example) or semisolid phase detection or a method based on a lateral flow system. Similar systems are already known, for example for rapid diagnosis of group A beta-hemolytic streptococcal pharyngitis (Streptatest).

Like mass spectrometry, this method makes it possible to detect the presence of Trichophytons with high sensitivity and high specificity. Advantageously, this method can be employed by anyone and allows a result to be obtained quickly, generally in 15 minutes.

For carrying out this method, the invention also relates to monoclonal antibodies directed against dipeptidyl-peptidase V, subtilisin-like protease 6 or leucine aminopeptidase 2 or a fragment of these antibodies capable of binding respectively to dipeptidyl-peptidase V, subtilisin-like protease 6 or leucine aminopeptidase 2. These antibodies are obtained by classical methods based on inoculating a rodent with, for example, dipeptidyl-peptidase V, subtilisin-like protease 6 or leucine aminopeptidase 2. The proteins or fragments of these purified or recombinant proteins obtained by genetic engineering are used as immunogens.

According to a last aim, the invention relates to a kit for diagnosing a disease of the skin and/or of its appendages linked to an infection with at least one Trichophyton, comprising at least one monoclonal antibody directed against dipeptidyl-peptidase V, subtilisin-like protease 6 or leucine aminopeptidase 2 or fragment of this antibody capable of binding to dipeptidyl-peptidase V, subtilisin-like protease 6 or leucine aminopeptidase 2, respectively. According to a particularly suitable embodiment, it is a kit for rapid diagnosis of onychomycosis.

The invention is now described with examples of nonlimiting tests, illustrating the invention and showing in particular its high specificity with respect to detection of the presence of Trichophytons in infected subjects. Certain tests were performed by mass spectrometry and others by immuno-detection based on samples of nail or of skin.

I. Protocols for Analysis of the Samples Applicable to the Various Tests

I.1 Extraction of the Proteins

The samples of healthy human skin are obtained by biopsy and constitute a negative control for verifying absence of DPPV.

The samples from human nails are obtained by scraping the nail bed of patients with onychomycoses or with other onychopathies.

For each sample: the proteins were extracted and solubilized in a solution containing 50 mM of ammonium bicarbonate and 0.1% of nonionic detergent ALS-400 (ref. Proteabio), which is compatible with analysis by mass spectrometry. Extraction was carried out with stirring in a Thermo mixer (Eppendorf) at a speed of 1400 rpm and at 4° C. for 2 hours. The supernatant containing the solubilized proteins was recovered by centrifugation (at 14000 rpm, at 4° C., for 10 minutes). The insoluble debris were removed by filtration through a 0.22-μm filter (Millipore, Billerica, United States, ref: UFC30GVNB), by centrifugation at 14000 rpm, at 4° C., for 10 minutes.

I.2 Immuno-Detection: Western Blot

The Western blot analyses are carried out after separation of the proteins in SDS-PAGE, according to the following protocol:

1. Transfer the proteins from the gel onto a nitrocellulose membrane with the iBlot™ Gel Transfer Device system (Invitrogen, US).

2. Humidify the membrane with PBS 1× for 5 minutes.

3. Block the nonspecific sites of the membrane with the Odyssey blocking buffer for one hour at room temperature (0.4 ml/cm²).

4. Dilute the primary antibody (total serum anti-proteases) in the Odyssey blocking buffer (LI-COR Biosciences, Germany) at 1:3000. Incubate overnight, stirring gently.

5. Wash the membrane with PBS 1× containing 0.1% of Tween-20 for 10 minutes. (3 successive washings).

6. Dilute the secondary antibody labeled with IRDye 800CW at 1:5000 in the Odyssey blocking buffer protected from the light.

7. Incubate for 60 minutes, protected from the light

8. Wash the membrane, protected from the light, with PBS 1× containing 0.1% of Tween-20 for 10 minutes. 3 washings.

9. Wash the membrane, protected from the light, with PBS 1× in order to remove the Tween-20.

10. Scan the membrane for infrared detection (Odyssey LI-COR, Germany).

I.3 Mass Spectrometry

The mass spectrometry analyses were performed with a triple quad, QSTAR® XL (APPLIED BIOSYSTEMS), essentially in MS/MS mode coupled upstream to a nano-HPLC.

Sample preparation for mass spectrometry analysis comprises a step of reduction and alkylation of the disulfide bridges of the proteins, followed by a step of enzymatic digestion (trypsin currently being the most used). The peptides obtained by digestion are then analyzed by mass spectrometry.

Enzymatic digestion may take place either directly in the gel: “in-gel digestion”, or in solution. With “in-gel digestion”, the proteins are separated beforehand in SDS-PAGE. In contrast, digestion in solution makes it possible to digest all the proteins starting from one and the same protein fraction. This method is generally used in cases when the samples are not very complex or are incompatible with separation in SDS-PAGE. The combination of SDS-PAGE and in-gel digestion shows the advantage of denaturing and separating the proteins and thus limiting the influence of the proteins that are very abundant, which, through steric hindrance, interfere with mass spectrometry analysis.

The choice of the method of digestion mainly depends on the quantity and complexity of the sample to be analyzed.

Direct Digestion in Solution:

For the protein samples, reduction followed by alkylation of the disulfide bridges was carried out prior to digestion by trypsin. This step was carried out using a reducing agent, dithiothreitol (DTT, Sigma Aldrich) and an alkylating agent, iodoacetamide. First, the clinical samples were incubated in a buffer solution of 100 mM of ammonium bicarbonate in the presence of 10 mM of dithiothreitol at 56° C. for 45 minutes with stirring, and then incubated with 55 mM of iodoacetamide for 30 minutes at room temperature, protected from the light.

Trypsin was used in order to “cut” the protein or proteins into peptides, which will then be analyzed by mass spectrometry.

Trypsin specifically hydrolyzes the peptide bond of the C-terminal end after the amino acids lysine and arginine (Lys-|-Xaa or Arg-|-Xaa) if the next amino acid is not a proline. The amount of trypsin was used in ratios (enzyme/substrate) between 1/10 and 1/100 in order to avoid autolysis of the trypsin when the concentration of enzyme is too high or incomplete hydrolysis of the proteins when the trypsin concentration is too low.

Digestion in solution by trypsin was carried out in an enzyme/protein ratio between 1/100 and 1/10 based on a solution of trypsin (1 mg/ml) diluted beforehand in 1 mM HCl solution. Incubation was carried out at 37° C. overnight with stirring.

“In-Gel” Digestion:

Prior to “in-gel” digestion, reduction followed by alkylation of the disulfide bridges in the clinical samples was carried out according to the protocol described in the section “Direct digestion in solution”. “In-gel” digestion proceeded according to the following steps:

1. The strips of interest were cut from a gel, in equal sections with a size of 1-1.5 mm using a scalpel, taking care to include only the stained gel.

2. The pieces of gel cut out were placed in a 2-ml plastic tube (Eppendorf, France).

3. The pieces of gel were covered with 200 μl of 120 mM ammonium bicarbonate with 40% of acetonitrile (ACN) and then were incubated at 37° C. for 30 minutes. The solutions were removed.

4. Step 3 was repeated once.

5. The pieces of gel were dried with the SpeedVac (Eppendorf, France) for 15 minutes.

6. The trypsin solution (0.6 μg) was added to the tube containing the pieces of gel. The amount of enzyme added was adjusted in order to cover the gel.

7. About 50 μl of 40 mM ammonium bicarbonate containing 9% of ACN was added to the tube to cover the pieces of gel.

8. Incubation was carried out at 37° C. overnight.

9. After incubation, the solution containing the peptides was transferred to a new tube.

10. 50 μl of the 50% ACN solution containing 0.1% formic acid was added to the tube containing the pieces of gel at 37° C. for 30 minutes with stirring. The supernatant was then combined with the solution recovered in step 9. This step was repeated.

11. The solution containing the extracted peptides was filtered by centrifugation at 1750 rpm for 2 minutes using an Ultrafree-MC 0.22 μm filter (Millipore, United States) to remove the residual pieces of gel.

12. The filtrate recovered in step 11 was evaporated in the SpeedVac (Eppendorf Concentrator 5301, France) for 60 minutes.

13. 20 μl of the 3% ACN solution containing 0.1% formic acid was added to the tube and then the solution was transferred to a special tube for analysis by mass spectrometry.

I-4 Nano HPLC-QSTAR® XL (MS/MS) Coupling

The HPLC system (LC Packing/DIONEX) is made up of an Ultimate pump equipped with a divider for delivering a nano flow rate (of the order of 200 nanoL/min), with a Famos sample injector (also configurable as a 96-well plate) and with a Switchos pump allowing preconcentration of the samples as well as removal of the salts that might interfere with detection by mass spectrometry.

The mobile phase is composed of a phase A (95 volumes of water, 5 volumes of ACN and 0.1 volume of formic acid) and a phase B (95 volumes of ACN, 5 volumes of water and 0.1 volume of formic acid) with a flow rate of 200 nL/min following a gradient for separation of the peptides. Chromatographic separation is performed on an Atlantis C18, Waters column (75 μm×15 cm) with a granulometry of 3 μm.

Preconcentration of the sample is carried out on a C18 LcPaking column with a length of 5 mm, diameter of 300 μm and a granulometry of 5 μm. The mobile phase for preconcentration is composed of a phase C (97 volumes of water, 3 volumes of ACN and 0.1 volume of formic acid) with a flow rate of 30 μL/min. Switching of the valve takes place 3 min after injection. The injection volume is 1 μL, but may be increased to 10 μL, depending on the type of analysis.

Mass Spectrometry:

The mass spectrometer is made up of a nano electrospray source (nanoESI), with the needle voltage between 1000 and 2000 V, set during calibration. The resolution is between 11000 and 13000 at mass 879.9 amu (atomic mass unit) (during calibration), the level of curtain gas is 30. All the spectra were acquired in positive ion reflector mode.

The quadrupole analyzer was used for effecting fragmentation in the argon collision cell. Fragmentation is performed for ions larger than 400 amu and smaller than 1200 amu with a state of charge between 2 and 3 amu and whose abundance is above 10 counts. These ions are excluded for 20 seconds after acquisition and for a window of 4.0 amu, the number of scans acquired for each MS/MS is 1 or 2. The duration of the runs varies from 80 min to 90 min depending on the samples, and quality controls are carried out roughly every 10 runs by injections of digest of glutamate dehydrogenase at a concentration of 50 femtomol. If the digest of glutamate dehydrogenase is identified on more than 5 significant peptides using Mascot software, the analyses are considered to be valid.

Analyst QS 1.0 software (Applied Biosystems) was used for data acquisition and for control of the instruments.

II. Tests Performed by Immuno-Detection

Tests were carried out for demonstrating the specificity of detection of the useful proteases according to the invention, in particular versus other proteases known to be secreted by a Trichophyton (Trichophyton rubrum) and that may be present in the samples.

These tests were performed by Western Blot with samples of:
nails infected with Trichophyton rubrum in persons with onychomycosis, and
injured nails.
   The proteases tested for are:

dipeptidyl-peptidase IV (DDPIV),

dipeptidyl-peptidase V (DDPV)

subtilisin-like protease 3 (SUB3),

subtilisin-like protease 4 (SUB4),

subtilisin-like protease 6 (SUB6),

subtilisin-like protease 7 (SUB7),

leucine aminopeptidase 2 (LAP2), and

metallocarboxypeptidase (M14A)

The results obtained are presented in the following table:

| | Detected in Western blot | |
|---|---|---|
| | in the nails infected with | Detected in Western blot |
| Proteases | T. rubrum | in the injured nails |
| DPPIV | NO | NO |
| DPPV | YES | NO |
| SUB3 | NO | NO |
| SUB4 | NO | NO |
| SUB6 | YES | NO |
| SUB7 | YES | NO |
| LAP2 | YES | NO |
| M14A | NO | NO |

These results show that only the proteases DPPV, SUB6, SUB7, and LAP2 are detected by Western blot in the nails infected with a Trichophyton.

Moreover, it can be seen that these proteases are not present in the injured nails, not infected with a Trichophyton.

Other tests were carried out to demonstrate the specificity of the presence of the proteases DPPV, SUB6, SUB7, and LAP2 at the level of the nails infected with a Trichophyton, versus other samples of skin or of nails not infected with a Trichophyton.

These tests were performed by Western blot with samples of:
nails infected with Trichophyton rubrum in persons with onychomycosis
healthy skin
stratum corneum of healthy human skin (surface layers of the epidermis)
injured nails and
nails affected by psoriasis.

The results obtained are presented in the following table:

| | Nails | Healthy | Healthy | | Psoriasis- |
|---|---|---|---|---|---|
| | infected with | skin | skin stratum | Injured | affected |
| Proteases | T. rubrum | (biopsies) | corneum | nails | nail |
| DPPV | YES | NO | NO | NO | NO |
| SUB6 | YES | NO | NO | NO | NO |
| SUB7 | YES | NO | NO | NO | NO |
| LAP2 | YES | NO | NO | NO | NO |

These results again show the specificity of the presence of the proteases DPPV, SUB6, SUB7, and LAP2 in samples of nails infected with a Trichophyton.

III. Tests Carried Out by Quantitative Mass Spectrometry

Supplementary tests were carried out to demonstrate the specificity of detection of the useful proteases according to the invention with another means of determination.

These tests were carried out by mass spectrometry with samples of:
nails infected with Trichophyton rubrum in persons with onychomycosis, and
injured nails.

The proteases tested for are:
dipeptidyl-peptidase IV (DDPIV),
dipeptidyl-peptidase V (DDPV),
subtilisin-like protease 3 (SUB3),
subtilisin-like protease 4 (SUB4),
subtilisin-like protease 6 (SUB6),
subtilisin-like protease 7 (SUB7),
leucine aminopeptidase 1 (LAP1),
leucine aminopeptidase 2 (LAP2), and
metallocarboxypeptidase (M14A).

The results obtained are presented in the following table:

| | Detected by mass | Detected by mass |
|---|---|---|
| | spectrometry in the nails | spectrometry in the |
| Proteases | infected with T. rubrum | injured nails |
| DPPIV | NO | NO |
| DPPV | YES | NO |
| SUB3 | NO | NO |
| SUB4 | NO | NO |
| SUB6 | YES | NO |
| SUB7 | YES | NO |
| LAP1 | YES | NO |
| LAP2 | YES | NO |
| M14A | NO | NO |

These results show that only the proteases DPPV, SUB6, SUB7, LAP2 and LAP1 are detected by mass spectrometry in the nails infected with a Trichophyton versus other proteases known to be secreted by a Trichophyton such as Trichophyton rubrum.

Moreover, it can be seen that these proteases DPPV, SUB6, SUB7, LAP2 and LAP1 are not present in the injured nails, not infected with a Trichophyton.

Other tests were carried out to demonstrate the specificity of the presence of one of the useful proteases according to the invention, namely SUB6, at the level of the nails infected with a Trichophyton, versus nails infected with other dermatophytes, presenting other diseases, or injured.

These tests were carried out by mass spectrometry with samples of nails presented in the following table:

| | Number of |
|---|---|
| Samples of nails | subjects |
| Trichophyton rubrum | 30 |
| Trauma (mycology negative) | 10 |
| Trichophyton rubrum + melanin | 2 |
| Trichophyton interdigitalis | 5 |
| Scytalidium dimidiatum | 4 |
| Fusarium | 2 |
| Aspergillus | 1 |
| Psoriasis Onychopathy (mycology negative) | 2 |
| Onychogryphosis (mycology negative) | 1 |

The sequences tested for by mass spectrometry reflecting the presence of SUB6 are:

SEQ ID No. 11: MANDVIQSPGEGTTGK
SEQ ID No. 12: VLDCDGSGSNSGVIK

The results obtained for detection of SUB6 in samples are presented in the following table:

| | SUB6 positive | |
|---|---|---|
| | (Number of | SUB6 positive |
| Samples of nails | subjects) | (%) |
| Trichophyton rubrum | 30 | 100 |
| Trauma (mycology negative) | 0 | 0 |
| Trichophyton rubrum + | 2 | 100 |
| melanin | | |
| Trichophyton interdigitalis | 0 | 0 |
| Scytalidium dimidiatum | 0 | 0 |
| Fusarium | 0 | 0 |
| Aspergillus | 0 | 0 |
| Psoriasis Onychopathy | 0 | 0 |
| (mycology negative) | | |
| Onychogryphosis (mycology | 0 | 0 |
| negative) | | |

These results confirm once again that detection of the protease SUB6 is specific to the presence of at least one Trichophyton in the sample tested.

IV—Immunodetection (ELISA) of Dipeptidyl-Peptidase V

The dipeptidyl-peptidase V specific to Trichophyton rubrum (Uniprot code of the sequence: Q9UW98) was obtained in the form of recombinant protein, and then purified.

SDS-PAGE analysis of the recombinant dipeptidyl-peptidase V after purification shows that this protein is in the form of a single nondegraded protein with apparent molecular weight of about 80 kDa.

The purified, nondenatured form of dipeptidyl-peptidase V served as antigen for producing monoclonal antibodies in the mouse according to a classical process (Eurogentec, France) according to the process described in https://secure.eurogentec.com/EGT/files/Pro-monoclon DEV-0409-V2.pdf; or according to the reference Kohler G, Milstein C., (1975) Continuous cultures of fused cells secreting antibody of predefined specificity. Nature. (5517): 495-7).

The clones are selected according to the protocol of the “antibody pair buffer kit” (InVitrogen, ref. CNB0011):

Briefly, MAXISORP plates are incubated with DPPV at a concentration of 2 μg/mL diluted in ASSAY BUFFER, depositing 50 μl per well. 3 h at 37° C., stirring at 300 rpm.

Wash the plate 4 times with WASH BUFFER, 200 μL/well.

Add the solution containing the test antibody diluted in the ASSAY BUFFER to 1:1000 at a rate of 50 μL per well and incubate for 2 hours at 37° C., stirring at 300 rpm.

Wash the plate 4 times with WASH BUFFER, 200 μL/well.

Add the secondary antibody coupled to horseradish peroxidase (rabbit antimouse) diluted with ASSAY BUFFER at a rate of 100 μL/well, and incubate for 30 min at room temperature, stirring at 300 rpm.

Wash the plate 4 times with WASH BUFFER, 200 μL/well.

Add 100 μL per well of enzyme substrate 3,3′,5,5′-tetramethyl benzidine (TMB) until a blue coloration appears and stop the reaction with 50 μL of 1M phosphoric acid (stop buffer) 15 to 30 min.

Read on the spectrophotometer at 450 nm.

Several clones were obtained in this way and characterized.

| Clones/Ig Isotypes | DPPV | Buffer | Delta OD |
|---|---|---|---|
| 6C4B12F12D9 | 0.941 | 0.048 | 0.893 |
| G1; Kappa | | | |
| 8H4B11B12C10 | 1.210 | 0.056 | 1.154 |
| G1; Kappa | | | |
| 4A10F5A7D9F9 | 1.209 | 0.051 | 1.158 |
| M; Kappa | | | |
| 4H6F11D12C12G11 | 1.328 | 0.049 | 1.279 |
| G3; Kappa | | | |
| 2G12B10C11 | 1.362 | 0.049 | 1.313 |
| G2a; Kappa | | | |
| Positive control | 0.571 | 0.055 | 0.516 |
| (serum) | | | |
| Blank | 0.130 | 0.053 | 0.077 |

Dipeptidyl-peptidase V (DPPV) was used at a concentration of 2 μg/mL. The difference in optical density (delta OD) between the clones put in contact with the protein DPPV and those put in contact with a buffer alone shows the immunoreactivity of the monoclonal antibodies. The positive control consists of the serum that was used for immunizing the mice to obtain the clones (at a dilution of 1/1000 in saturation buffer). The blank corresponds to the condition without clones.

A classical assay of the sandwich ELISA (enzyme-linked immunosorbent assay) type, based on the use of two different monoclonal antibodies and recombinant protein was used for identifying the conjugate-biotin pairs according to the protocol described in the “antibody pair buffer kit” from InVitrogen (ref. CNB0011):

Briefly, MAXISORP plates are incubated with the capture antibody (not biotinylated) in PBS at a concentration of 2 μg/mL overnight at 4° C. and 37° C.

Saturation of the plate with 150 μL of ASSAY BUFFER, then incubation for 3 h at 37° C., stirring at 300 rpm

Wash the plate 3 times with WASH BUFFER at a rate of 200 μL/well.

Add the antigen at a concentration of 4 μg/mL, 2 μg/mL, 0.5 μg/mL, 0.1 μg/mL, 0.05 μg/mL, 0.001 μg/mL, 0.005 μg/mL, 0 μg/mL, diluted in ASSAY BUFFER, deposit 50 μL per well.

Leave for 3 h at 37° C., stirring at 300 rpm.

Wash the plate 4 times with WASH BUFFER, 200 μL/well.

Add the solution of the 2nd antibody BUFFER, 200 μl/well.

Add the STREPAVIDIN-HRP solution at 1/1250th dilution with ASSAY BUFFER at a rate of 100 μL/well, and incubate for 30 min at room temperature, stirring at 300 rpm.

Wash the plate 4 times with WASH BUFFER, 200 μL/well.

Add 100 μL per well of enzyme substrate (TMB) until a blue coloration appears and stop the reaction with 50 μl of 1M phosphoric acid (stop buffer). 15 to 30 min.

Read on the spectrophotometer at 450 nm.

In the experiment presented below, the following sandwich ELISA protocol was adopted: The first “capture” antibody is absorbed on the bottom of the 96-well plate (Greiner) at a concentration of 2 μg/mL. The protein DPPV is used at a concentration of 0.2 μg/mL. The second antibody coupled to biotin (detection antibody) is used at a concentration of 0.01 μg/mL.

The blank is measured at OD=0.06; the control without dipeptidyl-peptidase V: OD=0.1 and the control without capture antibody at OD=0.07.

| | 6C4B12F12D9 | 8H4B11B12C10 | 4H6F11D12C12G11 | 2G12B10C11 |
|---|---|---|---|---|
| 6C4B12F12D9- | | 1.8 | 0.14 | 0.12 |
| biotin | | | | |
| 8H4B11B12C10- | 1.4 | | 0.12 | 0.12 |
| biotin | | | | |
| 4H6F11D12C12G11- | 0.45 | 0.26 | | 0.20 |
| biotin | | | | |
| 2G12B10C11- | 0.23 | 0.16 | 0.19 | |
| biotin | | | | |

The pair of antibodies comprising the clone 8H4B11B12C10 as capture antibody and the clone 6C4B12F12D9 as detection antibody is the most promising for carrying out the sandwich ELISA and was adopted.

The use of different concentrations of DPPV for setting up the sandwich ELISA made it possible to determine the lowest assayable concentration (see FIG. 1).

Based on the standard curve for ELISA detection of dipeptidyl-peptidase V, the limit of quantitation of dipeptidyl-peptidase V obtained is 0.0005 μg/ml. Blank: OD=0.11; DPPV 0.0005 μg/ml: OD=0.4

Dipeptidyl-peptidase V was assayed on various biological samples from nails infected with Trichophyton rubrum, but also of nails infected with other pathogens. Moreover, samples of nails showing a negative mycology (psoriasis, injury), as well as from healthy skin, were also used. Extraction of the proteins was carried out as described above.

Detection of DPPV in the biological samples was carried out as described for the standard range (using the purified recombinant protein DPPV). The total protein concentration in the biological samples was determined by Bradford's method. The concentration of DPPV obtained by assay and expressed in μg/mL was normalized relative to the total protein concentration determined by Bradford's method and expressed in 1 μg of DPPV/mg of total proteins.

| | | |
|---|---|---|
| | total | |
| DPPV | protein | DPPV |

| | μg/ml | [mg/ml] | ng/mg | ng/mg |
|---|---|---|---|---|
| T rubrum | 0.0444 | 3.43 | 13 | mean: 10 |
| | 0.0066 | 0.69 | 10 | |
| | 0.0010 | 0.14 | 8 | |
| T interdigitalis | 0.0293 | 1.64 | 18 | mean: 15 |
| | 0.0037 | 0.33 | 11 | |
| | 0.0010 | 0.07 | 15 | |

| | | | |
|---|---|---|---|
| Trauma | <0.0005 | 2.98 | Not detected |
| Scytalidium | <0.0005 | 1.79 | Not detected |
| dimidiatum | | | |
| Fusarium | <0.0005 | 1.52 | Not detected |
| Aspergillus | <0.0005 | 2.88 | Not detected |
| Psoriasis | <0.0005 | 0.18 | Not detected |
| Onychogryphosis | <0.0005 | 4.10 | Not detected |
| Healthy skin | <0.0005 | 2.63 | Not detected |

The data show good sensitivity of the ELISA as well as great specificity (only the samples containing Trichophyton are positive in ELISA).

1. A method of detecting the presence of at least one Trichophyton, the method comprising a step of determining the presence, in a sample of skin or skin appendage obtained from a human being or from an animal that can be infected with a Trichophyton, of at least one protease selected from the group consisting of dipeptidyl peptidase V, subtilisin-like protease 6, subtilisin-like protease 7, leucine aminopeptidase 1 and leucine aminopeptidase 2.

2. The method as claimed in claim 1, wherein the method is designed for detecting Trichophyton interdigitale and/or Trichophyton rubrum.

3. The method as claimed in claim 1, wherein the sample is a sample of nail obtained by scraping the nail bed or by piercing the nail using a microdrill.

4. The method as claimed in claim 1, for screening, therapeutic follow-up and/or diagnosis of a disease linked to an infection with at least one Trichophyton.

5. The method as claimed in one claim 1, for screening, therapeutic follow-up and/or diagnosis of an onychomycosis.

6. The method as claimed in claim 1, wherein the method comprises performing an immunologic test for determining the presence, in the sample, of at least one of the proteases selected from the group consisting of dipeptidyl peptidase V, subtilisin-like protease 6 and leucine aminopeptidase 2.

7. The method as claimed in claim 6, wherein the immunologic test comprises using at least one monoclonal antibody directed against dipeptidyl peptidase V, subtilisin-like protease 6 or leucine aminopeptidase 2 or a fragment of this antibody capable of binding respectively to dipeptidyl peptidase V, subtilisin-like protease 6 or leucine aminopeptidase 2.

8. The method as claimed in claim 1, wherein the method comprises a step of determining the presence of at least one of said proteases, performed by quantitative mass spectrometry.

9. The method as claimed in claim 8, wherein the step of determining the presence of at least one of said proteases comprises identifying, by quantitative mass spectrometry, at least one of the peptide sequences belonging to one of said proteases.

10. The method as claimed in claim 8, wherein the step of determining the presence of at least one of said proteases comprises identifying, by quantitative mass spectrometry, at least one of the following peptide sequences:
SEQ ID No. 1: LSVAEGVGLFNVLQEK
SEQ ID No. 2: ALVSHDGTFVGSSK
SEQ ID No. 3: GGVGIWISDAK
SEQ ID No. 4: INFVGYGQSTTK
SEQ ID No. 5: TLYVTAEDHATGK
SEQ ID No. 6: AAGAIVYNNVPGSLAGTLGGLDK
SEQ ID No. 7: VSFGIITDNVNANLTK
SEQ ID No. 8: LIVGFVTELAK
SEQ ID No. 9: HANAVNAMIATLSK
SEQ ID No. 10: KPGGTTYYYDPSAGK
SEQ ID No. 11: MANDVIQSPGEGTTGK
SEQ ID No. 12: VLDCDGSGSNSGVIK
SEQ ID No. 13: ADFSNYGAVVDVYAPGK
SEQ ID No. 14: SVMNMSLGGPR
SEQ ID No. 15: QMAIDVIQNPGASTTSK.

11. A monoclonal antibody directed against dipeptidyl peptidase V, subtilisin-like protease 6 or leucine aminopeptidase 2 or fragment of this antibody capable of binding respectively to dipeptidyl peptidase V, subtilisin-like protease 6 or leucine aminopeptidase 2, usable for carrying out the method as claimed in claim 1.

12. A kit for diagnosing a disease of the skin and/or of its appendages linked to an infection with at least one Trichophyton, wherein the kit comprises at least one monoclonal antibody as claimed in claim 11.

13. A kit for diagnosing onychomycosis, wherein the kit comprises at least one monoclonal antibody as claimed in claim 11.

14. At least one protease selected from the group consisting of dipeptidyl peptidase V, subtilisin-like protease 6, subtilisin-like protease 7, leucine aminopeptidase 1 and leucine aminopeptidase 2, for use thereof in a method of screening, of therapeutic follow-up and/or of diagnosis of a disease associated with an infection with at least one Trichophyton.

15. At least one protease selected from the group consisting of dipeptidyl peptidase V, subtilisin-like protease 6, subtilisin-like protease 7, leucine aminopeptidase 1 and leucine aminopeptidase 2, for use thereof in a method of screening, of therapeutic follow-up and/or of diagnosis of an onychomycosis.
